(19) United States
(12) Patent Application Publication
(10) Pub. No.: US 2006/0216246 A1
(43) Pub. Date: Sep. 28, 2006

(54) Protective oral gel

(75) Inventors: Eve Belanger, Saint Hyacinthe (CA); Elizabeth Marcu, Saint Hyacinthe (CA)

Correspondence Address: BORDEN LADNER GERVAIS LLP, WORLD EXCHANGE PLAZA, 100 QUEEN STREET SUITE 1100, OTTAWA, ON, K1P 1J9, CA

(73) Assignee: BioEnvelop Agro Inc.

(21) Appl. No.: 11/385,875

(22) Filed: Mar. 22, 2006

Related U.S. Application Data
(60) Provisional application No. 60/664,198, filed on Mar. 23, 2005.

(51) Int. Cl. A61K 8/73

(52) U.S. Cl. 424/49

(57) ABSTRACT

The present invention relates generally to protective barriers. More particularly, an aqueous oral gel is provided containing food-grade ingredients. The gel is used to protect or isolate soft tissue of the oral cavity, or teeth, during a dental procedure such as tooth whitening. The gel can be applied to a surface within the oral cavity as a liquid, and sets quickly to form a pliable protective barrier that can be carved away to expose a portion of a tooth, one or more teeth, or any area of the oral cavity on which the dental procedure is to be performed.

### CROSS REFERENCE TO RELATED APPLICATION

This application claims the benefit of and priority to U.S. patent application Ser. No. 60/664,198 filed Mar. 23, 2005, the entirety of which is herein incorporated by reference.

### FIELD OF THE INVENTION

The present invention relates generally to protective barrier devices. More particularly, the present invention relates to an aqueous oral gel for protecting or isolating tissue of the oral cavity.

### BACKGROUND OF THE INVENTION

Protective barrier devices for use during dental procedures are well known in the field. One such protective barrier device is the rubber dental dam. The positioning of the rubber dental dam inside the oral cavity can be cumbersome and often causes discomfort to the patient. The rubber dental dam is not suitable for protection of soft tissue or isolation of teeth during dental procedures requiring exposure to pastes and gels, such as fluoride treatments and tooth whitening.

Liquid rubber dams are also known in the art such as the Niveous Liquid Dam (Shofu Dental Corp.) and BriteSmile Barrier Material (BriteSmile Inc.). Such liquid rubber products comprise resins which are photo-polymerized, or light-cured, following application onto the gingival tissue.

Dental procedures such as fluoride treatment and tooth whitening are traditionally carried out by dispensing a paste or gel containing the active ingredient into an arch-shaped dental tray designed to fit over the surface of the upper and/or lower teeth. Dental trays may be manufactured in a standard arch shape, or may be custom-moulded to a patient's teeth for a better fit. Methods for customizing dental trays are known to those skilled in the art. Since tooth whitening requires prolonged exposure to potent oxidizing agents, such as peroxides, it is preferred to use a custom-moulded dental tray to reduce the amount of paste escaping from the dental tray, thereby coming into contact with adjacent soft tissue and potentially being swallowed by the patient.

One disadvantage of tooth whitening procedures involving the use of custom-moulded dental trays is that the method often requires two separate visits to the dental office: a first visit to prepare a cast from which the tray is moulded and a second visit to receive the treatment. It can take up to two weeks before the customized dental tray is prepared and ready to use. Furthermore, the customization of the tray adds to the expense of the procedure.

Efforts to reduce exposure of soft tissue to harsh dental compositions are worthwhile since certain dental procedures, such as tooth whitening procedures, can irritate and even damage the soft tissue of the oral cavity.

A standard dental tray could be employed for procedures such as tooth whitening, in order to reduce the time and expense of preparing a custom-moulded dental tray. However, for procedures such as tooth whitening, in which the oral cavity is exposed to harsh oxidizing agents over a prolonged period of time, using a standard dental tray alone does not offer protection to the adjacent soft tissue of the oral cavity. Further, a dental tray alone does not provide any barrier to adjacent teeth, for isolation, when only select teeth are to be the subject of the procedure.

### SUMMARY OF THE INVENTION

It is an object of the present invention to obviate or mitigate at least one disadvantage of the prior art by providing an aqueous oral gel that can be used to protect teeth or soft tissue during a dental procedure.

In accordance with a broad aspect of the present invention, there is provided an aqueous oral gel comprising, dextrin or dextrose, one or more plasticizing agents, lecithin, one or more gelling agents, modified cellulose, sucralose, acesulfame potassium, potassium chloride, and optionally, a non-toxic preservative.

In accordance with one embodiment of the invention, there is provided an aqueous oral gel comprising up to about 15% dextrin or dextrose, up to about 15% of one or more plasticizing agents, up to about 3% lecithin, up to about 6% of one or more gelling agents, up to about 10% modified cellulose, up to about 0.5% sucralose, up to about 0.5% acesulfame potassium, and up to about 2% potassium chloride. Optionally, the gel may include up to about 4% non-toxic preservative.

Further, the invention provides a method of protecting an oral cavity surface during a dental procedure comprising the steps of: applying the aqueous oral gel to the oral cavity surface in a liquid form at a temperature of from about 40° C. to about 50° C., and allowing the gel to set and form a pliable protective barrier.

In accordance with a further embodiment of the present invention, there is provided a method of protecting an oral cavity surface during a dental procedure comprising the steps of combining the following ingredients to a temperature of about 100° C. to provide a liquid formulation: up to about 15% dextrin or dextrose, up to about 15% of one or more plasticizing agents, up to about 3% lecithin, up to about 6% of one or more gelling agents, up to about 10% modified cellulose, up to about 0.5% sucralose, up to about 0.5% acesulfame potassium, up to about 2% potassium chloride, and water. Optionally, up to about 3% non-toxic preservative may be added. In a subsequent step, the liquid formulation is cooled to a temperature of from about 40° C. to about 50° C. to form a cooled liquid formulation, and the cooled liquid formulation is applied onto the oral cavity surface. The final step involves allowing the cooled liquid formulation to set and form a pliable protective barrier on the oral cavity surface.

Other aspects and features of the present invention will become apparent to those ordinarily skilled in the art upon review of the following detailed description of the embodiments of the invention.

### DETAILED DESCRIPTION

The invention provides an aqueous oral gel that can be used to protect or isolate an oral cavity surface during a procedure, such as a dental procedure.

The term “gel” refers to the formulation containing all of the ingredients indicated in any of its physical states, whether the formulation is in a set (semi-solid gelatinous) form, or in a liquid formulation (cooled or heated), which the gel may take at temperatures above room temperature, or at room temperature, but prior to setting.

The invention provides an aqueous oral gel comprising: up to about 15% dextrin or dextrose, up to about 15% of one or more plasticizing agents, up to about 3% lecithin, up to about 6% of one or more gelling agents, up to about 10% modified cellulose, up to about 0.5% sucralose, up to about 0.5% acesulfame potassium, and up to about 2% potassium chloride.

According to an embodiment, the aqueous oral gel comprises about 1.53% dextrin or dextrose, about 1.02% glycerol, about 0.85% carrageenan, about 0.76% lecithin, about 0.43% locust bean gum, about 1.04% modified cellulose, about 0.24% sucralose, about 0.08% acesulfame potassium, and about 0.20% potassium chloride.

Optionally, a flavour additive in an amount of up to about 2%. For example, mint flavour may be added in an amount of about 0.1%. The aqueous oral gel may further comprising a color additive.

A preservative may be included in the gel in an amount of up to 4%. The preservative may comprise up to 0.1% potassium sulfate, or may comprise, for example, about 3% lemon extract Biosecur™.

The plasticizing agent may comprise glycerol, sorbitol, or a combination of these. The gelling agent may be, for example, carrageenan, locust bean gum, agar agar, pectin, or a combination of these. The gelling agent may comprise kappa carrageenan in an amount of up to about 3% and locust bean gum in an amount of up to about 3%.

A method of protecting an oral cavity surface during a dental procedure is provided comprising the steps of: applying the aqueous oral gel of the invention to an oral cavity surface in a liquid form at a temperature of from about 40° C. to about 50° C., although higher or lower temperatures may be used, depending on the desired consistency and effect, and allowing the gel to set and form a pliable protective barrier. The additional step of carving away at least a portion of said barrier to expose an area designated to receive said dental procedure may also be included.

According to this method, the aqueous oral gel may comprise: about 1.53% dextrin or dextrose, about 1.02% glycerol, about 0.85% kappa carrageenan, about 0.76% de-oiled lecithin, about 0.43% locust bean gum, about 1.04% modified cellulose, about 0.24% sucralose, about 0.08% acesulfame potassium, and about 0.20% potassium chloride.

A method of protecting an oral cavity surface during a dental procedure is also provided comprising the steps of: combining the oral gel ingredients with water, and heating to a temperature of about 100° C. to provide a liquid formulation (the ingredients may optionally include a preservative in an amount of up to 4%); cooling said liquid formulation to a temperature of from about 40° C. to about 50° C. to form a cooled liquid formulation; applying said cooled liquid formulation onto the oral cavity surface, and allowing said cooled liquid formulation to set and form a pliable protective barrier on said oral cavity surface.

The method may also include the step of carving away at least a portion of said barrier to expose an area designated to receive said dental procedure.

A method of protecting an oral cavity surface during a dental procedure is provided, comprising application of the aqueous oral gel, disclosed herein, to an oral cavity surface to be protected.

In general, the protective oral gel of the present invention may include, in various non-toxic proportions, ingredients such as water, dextrin, dextrose, glycerol (glycerin), sorbitol, carrageenan, locust bean gum, agar agar, pectin, lecithin, soy lecithin, modified cellulose, carboxy-methyl cellulose, sucralose, acesulfame potassium, potassium chloride, and suitable non-toxic preservatives such as potassium sorbate. The oral gel of the present invention may further include flavoring and coloring. The term “non-toxic” is used in a general sense to indicate that topical application to living tissue will not have adverse effects.

A particularly advantageous embodiment of the gel of the present invention comprises: water, a dextrin/dextrose blend such as Moisture Lock™ (CK Food, Toronto, ON), glycerol, a carrageenan product containing sorbitol (Cambrian Chemicals, Inc, Oakville, ON), locust bean gum, lecithin, modified cellulose, sucralose, acesulfame potassium, and potassium chloride, with potassium sorbate added as a preservative. This particular embodiment is optionally provided with mint flavoring and blue coloring.

Table 1 shows the general proportions of ingredients found in the gel of the present invention. A skilled person will understand that certain variations and substitutions could be made without departing from the scope of the invention.

TABLE 1
Gel Ingredients

| INGREDIENT | AMOUNT (Percent w/w) |
|---|---|
| water | about 46-about 96% |
| lecithin | up to about 3% |
| potassium chloride | up to about 2% |
| plasticizing agent(s) | up to about 15% |
| sucralose | up to about 0.5% |
| acesulfame potassium | up to about 0.5% |
| dextrose, dextrin | up to about 15% |
| gelling agent(s)/gum(s) | up to about 6% |
| modified cellulose | up to about 10% |
| preservative* | from 0% to about 3% |
| TOTAL | 100% |

*Preservative is an optional ingredient

Soy lecithin may be used, or any other suitable lecithin source. One exemplary source may be dry soy lecithin that has been de-oiled.

As used herein, the term “plasticizing agent” refers to one or more substances that can provide to the gel the property of plasticizing, as may be commonly employed in food products. Exemplary plasticizing agents include glycerol (or glycerin), sorbitol, or combinations of these.

For the dextrin or dextrose component, either dextrin or dextrose may be used, or a blend of dextrin and dextrose together may be used. One such example of a dextrin/dextrose blend is Moisture Lock™ supplied by CK Foods (Toronto, ON).

As used herein, the term “gelling agent” refers to one or more substances that provide to the gel the property of gelling or congealing. Exemplary gelling agents include carrageenan, locust bean gum, agar agar, pectin (for example, low methoxy pectin), or combinations of these. Preferably the gelling agents are gums or fibers such as carrageenan (up to about 3%) and locust bean gum or agar agar (up to about 3%). Carrageenan in combination with sorbitol may be used, such as the product supplied by Cambrian Chemicals Inc. (Oakville, ON). An exemplary form of carrageenan may be kappa carrageenan.

Any type of modified cellulose may be used, such as carboxy-methyl cellulose.

For the optional preservative, any suitable non-toxic food-grade preservative such as potassium sorbate may be used. Potassium sorbate may be required in small quantities, for example of up to 0.1% w/w. Other preservatives may be used that are in a hydrated form such as a gel, in which case the weight of the gel would be considerably higher than the weight of a dry ingredient as a preservative. For example, a natural product preservative may be used, such as Biosecur™ made of lemon extract and containing limonoid glucosides and bioflavonoid in an aqueous medium (Biosecur Lab of Otterbum Park, Québec, Canada). Because a majority of the content of such a preservative is water, the weight contribution of this preservative ingredient to the final composition may be up to 4%, for example, 3%. A person skilled in the art can easily determine the appropriate quantity of such a preservative, based on the properties of the preservative, and the desired effect for the final composition of the gel.

There may be instances where no preservative is required, for example, if the solution is to be prepared and used within a set period of time that is not long enough to allow any significant microbial or fungal growth. The preservative is used in instances where the composition requires extended shelf life, storage at room temperature, to prevent contamination, to prevent microbial growth, or to promote stability over time.

The water content of the gel as a percentage of the total ingredients, is variable, depending on the amount of other ingredients to be included.

Table 2 shows the ingredient proportions of an exemplary embodiment of the aqueous gel of the present invention.

TABLE 2
Ingredients for an Embodiment of the Gel

| INGREDIENTS | AMOUNT (Percent w/w) |
|---|---|
| osmose water | 93.75% |
| lecithin (dry, de-oiled) | 0.76% |
| potassium chloride | 0.20% |
| glycerol (glycerin) | 1.02% |
| sucralose | 0.24% |
| acesulfame potassium | 0.08% |
| dextrose/dextrin¹ | 1.53% |
| carrageenan/sorbitol² | 0.85% |
| locust bean gum | 0.43% |
| modified cellulose³ | 1.04% |
| potassium sorbate⁴ | 0.1% |
| TOTAL | 100% |

¹a dextrin/dextrose blend, Moisture Lock ™ supplied by CK Food (Toronto, ON, Canada)
²A kappa carrageenan product containing sorbitol is used, supplied by Cambrian Chemicals Inc. (Oakville, ON, Canada)
³carboxy-methyl cellulose
⁴as an alternative to potassium sorbate, 3% Biosecur ™ could be used instead, with the water content of the gel being reduced by 2.9%.

The oral gel of the present invention is optionally provided with a flavour additive in an amount up to about 2%, preferably mint flavor additive in an amount of about 0.1%, but any flavor additive could be used. For example, bubble gum flavor can be used if the gel is intended for dental procedures in children.

The exemplary embodiment of the oral gel of the present invention is optionally provided with a color additive, preferably blue color, more preferably FD&C Blue no. 1, but any color could be used.

Advantageously, the basic components of the oral gel of the present invention are listed in Title 21 Code of Federal Regulations (CFR) as “Generally Recognized as Safe” (GRAS) and are approved for food use. Optional additives not considered GRAS, such as medicinal ingredients, can be added to the gel, in order to achieve a desired effect.

The non-toxic aqueous gel disclosed herein may also be applied to the skin to provide a protective barrier or to provide a spa treatment such as a facial mask or body wrap. The gel may be applied to the skin in liquid form at a temperature of about 40° C. to about 50° C. or any temperature which feels comfortable on the body. The nature of the gel is such that the liquid form when applied to the body at temperatures up to about 60° C. feels comfortable and will set within seconds of being applied. The term “set” is used to mean that gelling of the formulation occurs, converting the gel from a liquid to a gelatinous (semi-solid) state. The set gel can be peeled away from the underlying surface after it has set.

Because the gel is comfortable to the skin at relatively high temperatures, the gel is particularly advantageous when compared to other heated body wrap compositions, such as waxes, which may feel uncomfortable if applied to the body at 40° C. to 50° C. The gel of the present invention does not transfer heat to the skin or other biological surface (such as the oral cavity) in the way that conventional compositions can, and in this way there is no perception of a burning sensation when the inventive gel is applied in the temperature range of 40° C. to 50° C. The gel may be applied at a temperature below 40° C., and although the gel may have a slightly lumpy consistency at such lower temperatures, the function of the gel would not be impacted, as would be understood by a person skilled in the art. Temperatures about 50° C. can also be used, but depending on an individual's tolerance for such heat, there may be a slightly uncomfortable effect, but this does not impact the function of the gel.

The gel of the present invention may also be provided with an active ingredient, such as a topical anaesthetic, desensitizing agent, or UV protection agent, for direct application to a body or oral cavity surface. Any active ingredient is contemplated so long as the active ingredient does not diminish the barrier properties of the gel. As a spa treatment, active or therapeutic ingredients such as aromatherapy products may similarly be added.

Examples of oral cavity surfaces include but are not limited to surfaces of the teeth and soft tissue, including the gums, cheeks, hard and soft palate, and tongue, and also including the lips, oropharynx, and pharynx. The term “surface” is not intended to the limit the use of the oral gel to a single surface, but would encompass coverage of a plurality of surfaces within the oral cavity. It is contemplated that the gel may also be used to protect wounds or other openings within the oral cavity and may therefore penetrate into or beneath the exposed surface of the oral cavity.

The gel may be applied over a region within the oral cavity as a cooled liquid formulation, but prior to setting. In such an instance, the portion of the oral cavity to be exposed can be carved away prior to or during the procedure, for example by using a knife or other appropriate cutting tool.

Once the gel has set, a pliable protective barrier is formed. The barrier is pliable in that it can be moved or cut, but serves as a barrier to the portion of the oral cavity to which it is adjacent. The barrier can be protective in that chemicals or chemical solutions, UV or laser light, or other insults that may be employed during a procedure in the oral cavity are prevented from direct contact with the tissue adjacent to the gel.

The oral gel of the invention may be used to protect or isolate an oral cavity surface during various dental procedures, including but not limited to dental procedures such as tooth whitening, which generally involves applying a whitening paste or gel into the mouth. The term “dental procedure” is not intended to limit the use of the oral gel of the invention in any way. Any dental procedure, or procedure targeting the oral cavity, in which protection of a surface of the oral cavity is desired, is contemplated herein. The oral gel can also be used to isolate or protect teeth or soft tissue during oral surgery. The gel may be applied once prior to the procedure or may repeated as needed between steps during the procedure.

During certain procedures, it is desirable to designate only select teeth, or a portion of a select tooth, for the procedure, while excluding the remainder of the teeth. An advantage of the protective oral gel of the present invention is that is allows precise tailoring of the dental procedure to target only select teeth, or select portions of select teeth, that are designated for the procedure. As an example, consider a procedure in which an isolated stain is to be removed from a tooth surface through targeted whitening.

A further advantage of the inventive oral gel is that it can mitigate the use of custom-moulded dental trays for dental procedures such as tooth whitening. By eliminating the step of preparing a custom-moulded dental tray, the need for more than one visit to a dental office to complete a whitening procedure is eliminated. Alternatively, a standard (non-customized) tray could be employed during the procedure, since irritation and damage to soft tissue will be minimized by applying the protective gel over the gingival tissue prior to carrying out the procedure. A dental procedure such as tooth whitening could conveniently be carried out in a single visit to the dental office. Whether by mitigating the need to use a custom dental tray, or a dental tray altogether, or by permitting the use of a standard tray, the procedure is rendered more economical through use of the oral gel of the present invention.

The present invention provides an aqueous oral gel that is both non-toxic and edible, since the new gel eliminates the need for non-edible polymerizable resins. Advantageously, the components of the present invention when used as an oral gel are listed in Title 21 Code of Federal Regulations (FCR) as “Generally Recognized as Safe” (GRAS) and are approved for food use. Since protective barriers for use during dental procedures are applied into the oral cavity and retained therein for a prolonged period of time, there is an inherent risk that the patient may inadvertently ingest a portion, or all, of the protective barrier material, making it highly advantageous to use non-toxic food-grade materials.

The present invention advantageously provides an edible non-toxic aqueous oral gel, which provides a barrier for protecting soft tissue of the oral cavity or for isolating a tooth or a series of teeth from paste in the mouth during a dental procedure, such as tooth whitening. The protective oral gel is applied to a surface within the oral cavity in warm liquid form. The gel sets within seconds to form a pliable protective barrier that can be carved away to expose a portion of a tooth, an entire tooth, or a series of teeth on which the dental procedure, such a tooth whitening, is to be performed. The barrier can also be carved away to expose an area of soft tissue, such as gum, to be worked on during a procedure.

The non-toxic protective oral gel of the present invention is a food-grade quality gel that can be applied to any surface of the oral cavity prior to administration of a dental paste such as a tooth whitening paste. The protective gel is applied in the form of a warm liquid solution at a temperature of about 40° C. to about 50° C. and sets within seconds of being applied to a surface within the oral cavity to form a pliable gel barrier on said surface. A portion of the pliable gel barrier can then be carved away to expose the tooth or series of teeth on which the dental procedure is to be carried out. This warm liquid solution can also be referred to as the “cooled liquid form” of the gel, since the liquid composition is cooled to this temperature range, from a higher temperature, prior to being applied to oral cavity or onto any other biological surface.

In general, the aqueous oral gel of the present invention is prepared by mixing together a combination of non-toxic ingredients using water as the solvent. The term “aqueous” indicates that regardless of the exact proportions of ingredients used, or whether additional optional ingredients are added, the balance of the formulation is made up with water. The formulation is then heated to a temperature at which the mixture becomes a homogeneous solution, preferably to a temperature of about 100° C. for about 10 minutes for pasteurization. The formulation is then allowed to cool to a comfortable temperature at which the formulation can be applied to a surface within the oral cavity without causing discomfort to the subject. The formulation may be in a liquid or semi-liquid form as it is applied. Preferably the application temperature is in the range of about 40° C. to about 50° C. The term “semi-liquid” merely indicates that the setting process may begin as the liquid is being applied, depending on the application temperature and the time taken to carry out the application.

The gel composition of the present invention may be placed into a commercial package during manufacturing. The commercial packaging itself may include a box, a bag, a sealed pouch, a bottle, a combination thereof, or any other packaging suitable for distributing the product to a consumer. The product may thus be distributed or sold in the form of a commercial package comprising the oral gel of the invention together with instructions for use in any of the above-described applications.

### EXAMPLE 1

### Steps for Preparing Protective Oral Gel

Exemplary steps which may be taken for preparing a protective oral gel according to the present invention are provided below. The steps may be applied to ingredients listed above in Table 1 or Table 2. Ingredients may be combined in this manner or in a comparable manner that would accomplish a similar consistency of end-product.

1. Measure out water.

2. Add lecithin and mix to complete dissolution.

3. Add potassium chloride and mix to complete dissolution.

4. Add glycerol and mix for about 2 minutes.

5. Pre-mix sucralose and acesulfame potassium prior to adding and then mix to complete dissolution.

6. Add dextrose/dextrin and mix to complete dissolution.

7. Pre-mix carrageenan, locust bean gum and modified cellulose prior to adding and then mix to complete hydration.

8. Heat to about 100° C. and maintain temperature for about 10 minutes to accomplish pasteurization.

9. After pasteurization, add potassium sorbate.

10. Cool product to about 65° C. and then add flavor.

11. Let the product stand for a few hours prior to use.

### EXAMPLE 2

### Steps to Heat Gel in a Double Boiler

The following steps may be used to heat the gel of the invention to achieve a desired temperature, for example to heat to about 100° C. With reference to Example 1, these steps may be used to accomplish pasteurization.

1. Cut the desired quantity of the gel into pieces.

2. Place the product in a heat resistant container.

3. Place the product in a double-boiler. The product should be stirred frequently to disperse heat and prevent overheating. The product should be completely melted.

4. Cool the product solution to approximately 48-50° C. (120-122° F.). Stir frequently during the cooling step to obtain a solution as homogenous as possible.

5. The solution is ready to use.

### EXAMPLE 3

### Steps to Heat Gel in a Microwave

The following steps may be used to heat the gel of the invention to achieve a desired temperature, for example to heat to about 100° C. With reference to Example 1, these steps may be used to accomplish pasteurization.

1. Cut the desired quantity of the product into pieces.

2. Place the product in a heat resistant container.

3. Place the container in the microwave at a maximum power for 1 minute and then stir the solution. Repeat the process until the product is completely melted.

4. Cool the solution to approximately 48-50° C. (120-122° F.).

5. The solution is ready to use.

### EXAMPLE 4

### Steps to Apply the Gel in Liquid Form

Once the protective gel has been formed and is at an appropriate temperature in liquid form, application to an oral surface can be conducted as outlined below.

1. Load the solution into a small barrel syringe.

2. Express the material onto the gingival immediately adjacent to the crown portion of the tooth (dento-gingival interface) in an even manner.

3. The measured thickness should be approximately 2 mm following the tooth/gum line architecture.

4. A few seconds after application, the gel will form a soft barrier gel that can be carved in order to expose the portion of the tooth or teeth to be treated by a paste containing the active whitening ingredient.

### EXAMPLE 5

### Clinical Testing of the Protective Oral Gel

The protective gel outlined in Table 2 was prepared according to Example 1, and heated as outlined in Example 2. The gel was applied to the gingival tissue of over 35 subjects in Canada, who were then exposed to carbamide and chlorine dioxide gels, and to peroxide solutions which were applied to the teeth and gingival tissue. The protective gel according to the invention provided an effective barrier for protection of the gingival tissue.

All references referred to herein are incorporated by reference. The above-described embodiments of the present invention are intended to be examples only. Alterations, modifications and variations may be effected to the particular embodiments by those of skill in the art without departing from the scope of the invention, which is defined solely by the claims appended hereto.

1. An aqueous oral gel comprising:
up to about 15% dextrin or dextrose,
up to about 15% of one or more plasticizing agents,
up to about 3% lecithin,
up to about 6% of one or more gelling agents,
up to about 10% modified cellulose,
up to about 0.5% sucralose,
up to about 0.5% acesulfame potassium, and
up to about 2% potassium chloride.

2. The aqueous oral gel according to claim 1 comprising,
about 1.53% dextrin or dextrose,
about 1.02% glycerol,
about 0.85% carrageenan,
about 0.76% lecithin,
about 0.43% locust bean gum,
about 1.04% modified cellulose,
about 0.24% sucralose,
about 0.08% acesulfame potassium, and
about 0.20% potassium chloride.

3. The aqueous oral gel of claim 1 further comprising a flavour additive in an amount of up to about 2%.

4. The aqueous oral gel of claim 3 wherein the flavour additive comprises mint flavour in an amount of about 0.1%.

5. The aqueous oral gel of claim 1 further comprising a color additive.

6. The aqueous oral gel of claim 1 further comprising a preservative in an amount of up to 4%.

7. The aqueous oral gel of claim 6, wherein the preservative comprises up to 0.1% potassium sulfate.

8. The aqueous oral gel of claim 6, wherein the preservative comprises about 3% lemon extract Biosecur™.

9. The aqueous oral gel of claim 1 wherein said plasticizing agent comprises glycerol, sorbitol, or a combination of these.

10. The aqueous oral gel of claim 1 wherein said gelling agent is carrageenan, locust bean gum, agar agar, pectin, or a combination of these.

11. The aqueous oral gel of claim 1 wherein said gelling agent comprises kappa carrageenan in an amount of up to about 3% and locust bean gum in an amount of up to about 3%.

12. A method of protecting an oral cavity surface during a dental procedure comprising the steps of:
applying the aqueous oral gel of claim 1 to the oral cavity surface in a liquid form at a temperature of from about 40° C. to about 50° C., and
allowing the gel to set and form a pliable protective barrier.

13. The method of claim 12 further comprising the step of carving away at least a portion of said barrier to expose an area designated to receive said dental procedure.

14. The method of claim 12 wherein the aqueous oral gel comprises:
about 1.53% dextrin or dextrose,
about 1.02% glycerol,
about 0.85% kappa carrageenan,
about 0.76% de-oiled lecithin,
about 0.43% locust bean gum,
about 1.04% modified cellulose,
about 0.24% sucralose,
about 0.08% acesulfame potassium, and
about 0.20% potassium chloride.

15. A method of protecting an oral cavity surface during a dental procedure comprising the steps of:
combining the following ingredients to a temperature of about 100° C. to provide a liquid formulation:
up to about 15% dextrin or dextrose,
up to about 15% of one or more plasticizing agents,
up to about 3% lecithin,
up to about 6% of one or more gelling agents,
up to about 10% modified cellulose,
up to about 0.5% sucralose,
up to about 0.5% acesulfame potassium, and
up to about 2% potassium chloride,
from 0% to about 4% non-toxic preservative, and
water;
cooling said liquid formulation to a temperature of from about 40° C. to about 50° C. to form a cooled liquid formulation,
applying said cooled liquid formulation onto the oral cavity surface, and
allowing said cooled liquid formulation to set and form a pliable protective barrier on said oral cavity surface.

16. The method of claim 15 further comprising the step of carving away at least a portion of said barrier to expose an area designated to receive said dental procedure.

17. The method of claim 15 wherein the liquid formulation comprises:
about 1.53% dextrin or dextrose,
about 1.02% glycerol,
about 0.85% carrageenan,
about 0.76% lecithin,
about 0.43% locust bean gum
about 1.04% modified cellulose,
about 0.24% sucralose,
about 0.08% acesulfame potassium, and
about 0.20% potassium chloride.

18. A method of protecting an oral cavity surface during a dental procedure comprising application of the aqueous oral gel of claim 1 to an oral cavity surface to be protected.

19. A method of protecting an oral cavity surface during a dental procedure comprising application of the aqueous oral gel of claim 2 to an oral cavity surface to be protected.
